(19) United States
(12) Patent Application Publication
(10) Pub. No.: US 2023/0295211 A1
(43) Pub. Date: Sep. 21, 2023

(54) METHODS OF PRODUCING CRYSTALLINE BETA NICOTINAMIDE RIBOSIDE TRIACETATE CHLORIDE

(71) Applicant: ChromaDex Inc., Los Angeles, CA (US)

(72) Inventors: Aron Erickson, Longmont, CO (US); Philip Redpath, Firestone, CO (US); Jacob Roodman, Superior, CO (US); Richard Nygaard, Longmont, CO (US)

(73) Assignee: ChromaDex Inc., Los Angeles, CA (US)

(21) Appl. No.: 18/184,456

(22) Filed: Mar. 15, 2023

Related U.S. Application Data
(60) Provisional application No. 63/319,997, filed on Mar. 15, 2022.

(51) Int. Cl. C07H 19/048

(52) U.S. Cl. CPC C07H 19/048; C07B 2200/13

(57) ABSTRACT

This disclosure relates to a process for producing Crystalline Beta Nicotinamide Riboside Triacetate Chloride with improved physical property characteristics. A substantially crystalline Beta Nicotinamide Riboside Triacetate Chloride, or a salt, or a solvate thereof is described having a chemical purity of greater than about 90% (w/w) and containing less than about 5000 ppm ethanol.

This application claims the benefit of U.S. Provisional application No. 63/319,997, filed on Mar. 15, 2022, which is hereby incorporated by reference herein in its entirety.

### TECHNICAL FIELD

The present invention relates to a process for producing Crystalline Beta Nicotinamide Riboside Triacetate Chloride with improved physical property characteristics.

### BACKGROUND

Nicotinamide riboside (NR) is a valuable bioactive intermediate. This compound has been implicated in processing and metabolic pathways involving NAD+ (J. Preiss and P. Handler, J. Biol. Chem. (1958) 233:488-492).

The dietary vitamin B3, which encompasses nicotinamide (“Nam” or “NM”), nicotinic acid (“NA”), and nicotinamide riboside (“NR”), is a precursor to the coenzyme nicotinamide adenine dinucleotide (“NAD⁺”), its phosphorylated parent (“NADP⁺” or “NAD(P)⁺”), and their respective reduced forms (“NADH” and “NADPH,” respectively). Once converted intracellularly to NAD(P)⁺ and NAD(P)H, vitamin B3 metabolites are used as co-substrates in multiple intracellular protein modification processes, which control numerous essential signaling events (e.g., adenosine diphosphate ribosylation and deacetylation), and as cofactors in over 400 redox enzymatic reactions, thus controlling metabolism. This is demonstrated by a range of metabolic endpoints, which include the deacylation of key regulatory metabolic enzymes, resulting in the restoration of mitochondrial activity and oxygen consumption. Critically, mitochondrial dysfunction and cellular impairment have been correlated to the depletion of the NAD(P)(H)-cofactor pool, when the NAD(P)(H)-cofactor pool is present in sub-optimal intracellular concentrations. Vitamin B3 deficiency yields to evidenced compromised cellular activity through NAD(P)⁺ depletion, and the beneficial effect of additional NAD(P)⁺ bioavailability through NA, Nam, NR, and nicotinamide mononucleotide (“NMN”) supplementation is primarily observed in cells and tissues where metabolism and mitochondrial function have been compromised.

Despite extensive optimization of solution-based methodologies over many years for nucleotide preparation, difficulties and issues remain in the syntheses of nicotinoyl ribosides, the monophosphorylation of active hydroxyl groups thereof, and subsequent conjugation thereof, with respect to low yields and product stability and isolation from polar solvents. The current methodologies are also plagued by atom and energy inefficiency due, for example, to the use of large solvent excesses and the need for temperature-controlled reaction conditions.

The reported syntheses of nicotinamide riboside (NR) are becoming more scalable, but use corrosive and expensive reagents, and lengthy deprotection steps, and thus still display batch-to-batch quality variation, thereby presenting difficulties in maintaining good standards.

Partially protected nucleosides and nucleotides have found broad-ranging application in order to achieve improved bioavailability of the nucleoside and nucleotide parents. Such partial protection includes hydroxyl modifications with ester, carboxylate, and acetyl groups, in addition to the introduction of hydrolyzable phosphoramidate or mixed anhydride modification of the phosphate monoesters in the form of Protides and CycloSal derivatives. While the former type of protection has become more scalable, the modifications at the phosphorus center remain difficult to accomplish at scale, particularly on nucleosidic entities that are highly sensitive to changes in pH and that are readily degraded by heat.

Reduced nicotinamide riboside (“NRH”) has been consistently shown to be more efficient at increasing intracellular NAD⁺ levels, and surpasses nicotinamide riboside (NR) in that respect. While physiological and potentially therapeutic roles have not yet been examined due to a lack of material accessible in sufficient quantities for broad-ranging studies, it is anticipated that the phosphorylated forms of NRH and reduced nicotinic acid riboside (“NARH”), or derivatives thereof, could also have similar NAD⁺-boosting capacities.

The reported syntheses of reduced nicotinamide riboside (NRH) are becoming more widely available but remain conducted on small scales, using corrosive and expensive reagents, and lengthy deprotection steps, and thus still display batch-to-batch quality variation, thereby presenting difficulties in maintaining good standards. In the current description, reduced nicotinamide riboside (NRH) generally refers to “reduced pyridine” nucleus, more specifically, the 1,4-dihydropyridine compounds.

Synthetically, the preparation of 5′-nucleotides remains time-consuming, atom-inefficient, and costly, due to the need for numerous protection and deprotection steps. In these preparation methods, the chlorodialkylphosphate, tetraalkylpyrophosphate, chlorophosphite, or phosphoramidite reagents required are also expensive starting materials by virtue of their chemical functionalization and chemical instability, and therefore, consequently associated synthetic difficulties. Phosphorylation reaction conditions are difficult to control and often use non-approved or toxic organic solvents, thus limiting the market of the manufactured compounds.

One known alternative approach to the protection/deprotection method is to use phosphorus oxychloride (P(O)Cl₃) (i.e., Yoshikawa conditions), however there are still drawbacks to this method, as follows. While not being bound by theory, in this method, polar trialkyl phosphate solvents, such as P(O)(OMe)₃, are used in a large excess, which are believed to enhance reaction rates while limiting the undesirable reactivity of P(O)Cl₃ as a chlorinating agent. Thus, it is believed that use of excess P(O)Cl₃/P(O)(OR)₃ is a better combination for the chemoselective 5′-O-phosphorylation of unprotected ribosides. However, the use of trialkyl phosphate solvents, such as P(O)(OMe)₃, precludes their implementation for the preparation of materials for eventual human use, as this class of solvent is highly toxic (known carcinogen, non-GRAS approved) and is difficult to remove from the final polar products. See M. Yoshikawa et al., Studies of Phosphorylation. III, Selective Phosphorylation of Unprotected Nucleosides, 42 BULL. CHEM. SOC. JAPAN 3505 (1969); Jaemoon Lee et al., A chemical synthesis of nicotinamide adenine dinucleotide (NAD+), CHEM. COMMUN. 729 (1999); each of which is incorporated by reference herein in its entirety.

Nicotinamide adenine dinucleotide (NAD⁺) remains an expensive cofactor, and its commercial availability is simply limited by its complex chemical nature and the highly reactive pyrophosphate bond, which is challenging to form at scale.

Nicotinoyl ribosides such as nicotinamide riboside (NR) and nicotinic acid riboside (“NAR”), nicotinamide mononucleotide (NMN), and NAD⁺ are viewed as useful bioavailable precursors of the NAD(P)(H) pool to combat and treat a broad range of non-communicable diseases, in particular those associated with mitochondrial dysfunction and impaired cellular metabolism. Optimizing the large-scale syntheses of these vitamin B3 derivatives is therefore highly valuable to make these compounds more widely available to society both in terms of nutraceutical and pharmaceutical entities.

Reduced nicotinoyl ribosides, such as reduced nicotinamide riboside (NRH), reduced nicotinic acid riboside (NARH), reduced nicotinamide mononucleotide (“NMNH”), reduced nicotinic acid mononucleotide (“NaMNH”), and reduced nicotinamide adenine dinucleotide (“NADH”) are viewed as useful bioavailable precursors of the NAD(P)(H) pool to combat and treat a broad range of non-communicable diseases, in particular those associated with mitochondrial dysfunction and impaired cellular metabolism. Optimizing the large-scale syntheses of these vitamin B3 derivatives is therefore highly valuable to make these compounds more widely available to society, both in terms of nutraceutical and pharmaceutical entities.

Crystalline forms of useful molecules can have advantageous properties relative to the respective amorphous forms of such molecules. For example, crystal forms are often easier to handle and process, for example, when preparing compositions that include the crystal forms. Crystalline forms typically have greater storage stability and are more amenable to purification. The use of a crystalline form of a pharmaceutically useful compound can also improve the performance characteristics of a pharmaceutical product that includes the compound. Obtaining the crystalline form also serves to enlarge the repertoire of materials that formulation scientists have available for formulation optimization, for example by providing a product with different properties, e.g., better processing or handling characteristics, improved dissolution profile, or improved shelf-life.

WO 2016/014927 A2, incorporated by reference herein in its entirety, describes crystalline forms of nicotinamide riboside, including a Form I of nicotinamide riboside chloride. Also disclosed are pharmaceutical compositions comprising the crystalline Form I of nicotinamide riboside chloride, and methods of producing such pharmaceutical compositions.

WO 2016/144660 A1, incorporated by reference herein in its entirety, describes crystalline forms of nicotinamide riboside, including a Form II of nicotinamide riboside chloride. Also disclosed are pharmaceutical compositions comprising the crystalline Form II of nicotinamide riboside chloride, and methods of producing such pharmaceutical compositions.

In view of the above, there is a need for processes that are atom-efficient in terms of reagent and solvent equivalency, that bypass the need for polar, non-GRAS (“generally recognized as safe”) solvents, that are versatile in terms of limitations associated with solubility and reagent mixing, that are time- and energy-efficient, and that provide efficient, practical, and scalable methods for the preparation of nicotinoyl ribosides, reduced nicotinoyl ribosides, modified derivatives thereof, phosphorylated analogs thereof, and adenylyl dinucleotide conjugates thereof.

In view of the above, there is a need for novel crystalline forms of nicotinoyl ribosides, reduced nicotinoyl ribosides, modified derivatives thereof, phosphorylated analogs thereof, and adenylyl dinucleotide conjugates thereof.

Nicotinic acid and nicotinamide, collectively niacins, are the vitamin forms of nicotinamide adenine dinucleotide (NAD+). Eukaryotes can synthesize NAD+ de novo via the kynurenine pathway from tryptophan (Krehl, et al. Science (1945) 101:489-490; Schutz and Feigelson, J. Biol. Chem. (1972) 247:5327-5332) and niacin supplementation prevents the pellagra that can occur in populations with a tryptophan-poor diet. Thus, it is well-established that nicotinic acid is phosphoribosylated to nicotinic acid mononucleotide (NaMN), which is then adenylylated to form nicotinic acid adenine dinucleotide (NaAD), which in turn is amidated to form NAD+ (Preiss and Handler (1958) 233:488-492; Ibid., 493-50).

Nicotinamide Adenine Dinucleotide (“NAD⁺”) is an enzyme co-factor that is essential for the function of several enzymes related to reduction-oxidation reactions and energy metabolism. (Katrina L. Bogan & Charles Brenner, Nicotinic Acid, Nicotinamide, and Nicotinamide Riboside: A Molecular Evaluation of NAD⁺ Precursor Vitamins in Human Nutritions, 28 Annual Review of Nutrition 115 (2008)). NAD⁺ functions as an electron carrier in cell metabolism of amino acids, fatty acids, and carbohydrates. (Bogan & Brenner 2008). NAD⁺ serves as an activator and substrate for sirtuins, a family of protein deacetylases that have been implicated in metabolic function and extended lifespan in lower organisms. (Laurent Mouchiroud et al., The NAD⁺/Sirtuin Pathway Modulates Longevity through Activation of Mitochondrial UPR and FOXO Signaling, 154 Cell 430 (2013)). The co-enzymatic activity of NAD⁺, together with the tight regulation of its biosynthesis and bioavailability, makes it an important metabolic monitoring system that is clearly involved in the aging process.

Once converted intracellularly to NAD(P)⁺, vitamin B3 is used as a co-substrate in two types of intracellular modifications, which control numerous essential signaling events (adenosine diphosphate ribosylation and deacetylation), and is a cofactor for over 400 reduction-oxidation enzymes, thus controlling metabolism. This is demonstrated by a range of metabolic endpoints including the deacetylation of key regulatory proteins, increased mitochondrial activity, and oxygen consumption. Critically, the NAD(P)(H)-cofactor family can promote mitochondrial dysfunction and cellular impairment if present in sub-optimal intracellular concentrations. Vitamin B3 deficiency yields to evidenced compromised cellular activity through NAD depletion, and the beneficial effect of additional NAD bioavailability through nicotinic acid (“NA”), nicotinamide (“Nam”), and nicotinamide riboside (“NR”) supplementation is primarily observed in cells and tissues where metabolism and mitochondrial function had been compromised.

Interestingly, supplementation with nicotinic acid (“NA”) and nicotinamide (“Nam”), while critical in acute vitamin B3 deficiency, does not demonstrate the same physiological outcomes compared with that of nicotinamide riboside (“NR”) supplementation, even though at the cellular level, all three metabolites are responsible for NAD biosynthesis. This emphasizes the complexity of the pharmacokinetics and bio-distribution of B3-vitamin components.

The bulk of intracellular NAD is believed to be regenerated via the effective salvage of nicotinamide (“Nam”) while de novo NAD is obtained from tryptophan. (Anthony Rongvaux et al., Reconstructing eukaryotic NAD metabolism, 25 BioEssays 683 (2003)). Crucially, these salvage and de novo pathways apparently depend on the functional forms of vitamins B1, B2, and B6 to generate NAD⁺ via a phosphoriboside pyrophosphate intermediate. Nicotinamide riboside (“NR”) is the only form of vitamin B3 from which NAD⁺ can be generated in a manner independent of vitamins B1, B2, and B6, and the salvage pathway using nicotinamide riboside (“NR”) for the production of NAD⁺ is expressed in most eukaryotes.

The main NAD⁺ precursors that feed the salvage pathways are nicotinamide (“Nam”) and nicotinamide riboside (“NR”). (Bogan & Brenner 2008). Studies have shown that nicotinamide riboside (“NR”) is used in a conserved salvage pathway that leads to NAD⁺ synthesis through the formation of nicotinamide mononucleotide (“NMN”). Upon entry into the cell, nicotinamide riboside (“NR”) is phosphorylated by the NR kinases (“NRKs”), generating NMN, which is then converted to NAD by nicotinamide mononucleotide adenylyltransferase (“NMNAT”). (Bogan & Brenner 2008). Because NMN is the only metabolite that can be converted to NAD⁺ in mitochondria, nicotinamide (“Nam”) and nicotinamide riboside (“NR”) are the two candidate NAD⁺ precursors that can replenish NAD⁺ and thus improve mitochondrial fuel oxidation. A key difference is that nicotinamide riboside (“NR”) has a direct two-step pathway to NAD⁺ synthesis that bypasses the rate-limiting step of the salvage pathway, nicotinamide phosphoribosyltransferase (“NAMPT”). Nicotinamide (“Nam”) requires NAMPT activity to produce NAD⁺. This reinforces the fact that nicotinamide riboside (“NR”) is a very effective NAD⁺ precursor. Conversely, deficiency in dietary NAD⁺ precursors and/or tryptophan causes pellagra, a disease characterized by dermatitis, diarrhea, and dementia. (Bogan & Brenner 2008). In summary, NAD⁺ is required for normal mitochondrial function, and because mitochondria are the powerhouses of the cell, NAD⁺ is required for energy production within cells.

NAD+ was initially characterized as a co-enzyme for oxidoreductases. Though conversions between NAD+, NADH, NADP and NADPH would not be accompanied by a loss of total co-enzyme, it was discovered that NAD+ is also turned over in cells for unknown purposes (Maayan, Nature (1964) 204:1169-1170). Sirtuin enzymes such as Sir2 of S. cerevisiae and its homologs deacetylate lysine residues with consumption of an equivalent of NAD+ and this activity is required for Sir2 function as a transcriptional silencer (Imai, et al., Cold Spring Harb. Symp. Quant. Biol. (2000) 65:297-302). NAD+-dependent deacetylation reactions are required not only for alterations in gene expression but also for repression of ribosomal DNA recombination and extension of lifespan in response to calorie restriction (Lin, et al., Science (2000) 289:2126-2128; Lin, et al., Nature (2002) 418:344-348). NAD+ is consumed by Sir2 to produce a mixture of 2′- and 3′ O-acetylated ADP-ribose plus nicotinamide and the deacetylated polypeptide (Sauve, et al., Biochemistry (2001) 40:15456-15463). Additional enzymes, including poly(ADPribose) polymerases and cADPribose synthases are also NAD+-dependent and produce nicotinamide and ADPribosyl products (Ziegler, Eur. J. Biochem. (2000) 267:1550-1564; Burkle, Bioessays (2001) 23:795-806).

U.S. Pat. No. 9,975,915, incorporated by reference herein in its entirety, describes crystalline forms of nicotinamide riboside, including a NR methanolate Form II of nicotinamide riboside chloride. Also disclosed are compositions comprising the NR methanolate Form II of nicotinamide riboside chloride, and methods of preparation of the NR methanolate Form II of nicotinamide riboside chloride. Also disclosed are crystalline forms of nicotinic acid riboside (NAR), including a Form I of nicotinic acid riboside (NAR). Also disclosed are compositions comprising the Form I of nicotinic acid riboside (NAR), and methods of preparation of the Form I of nicotinic acid riboside (NAR). Also disclosed are crystalline forms of nicotinamide riboside triacetate (1-(2′,3′,5′-triacetyl-beta-D-ribofuranosyl)-nicotinamide, “NR triacetate,” or “NRTA”, a.k.a. “NRT”), including a Form I of nicotinamide riboside triacetate (NRTA) chloride (“NRTA-Cl”). Also disclosed are compositions comprising the Form I of nicotinamide riboside triacetate (NRTA), and methods of preparation of the Form I of nicotinamide riboside triacetate (NRTA). Also disclosed are crystalline forms of nicotinic acid riboside triacetate (1-(2′,3′,5′-triacetyl-beta-D-ribofuranosyl)-nicotinic acid, “NAR triacetate,” or “NARTA”), including a Form I of nicotinic acid riboside triacetate (NARTA). Also disclosed are compositions comprising the Form I of nicotinic acid riboside triacetate (NARTA), and methods of preparation of the Form I of nicotinic acid riboside triacetate (NARTA). Also disclosed are crystalline forms of nicotinamide mononucleotide (“NMN”), including a Form III of nicotinamide mononucleotide (NMN), and a Form IV of nicotinamide mononucleotide (NMN). Also disclosed are compositions comprising the Form III of nicotinamide mononucleotide (NMN) and compositions comprising the Form IV of nicotinamide mononucleotide (NMN), and methods of preparation of the Form III of nicotinamide mononucleotide (NMN) and methods of preparation of the Form IV of nicotinamide mononucleotide (NMN).

Nicotinamide Riboside Chloride is known to exist as two stable polymorphs, Form I and Form II. Known synthesis and purification procedures have shown to produce mixtures of Form I and Form II with poor physical properties presenting difficulties in downstream encapsulation processing.

Additionally, Nicotinamide Riboside Chloride Triacetate Chloride (NRTA-Cl) is known to exist as a stable polymorph, namely Form I. There are known difficulties in the large scale production of NRTA-C1, including identification of a scalable crystallization process.

The present invention attempts to solve these problems as well as others.

### SUMMARY OF THE INVENTION

The referenced invention provides process conditions shown to improve particle size distribution, bulk density, and polymorph control for the production of Nicotinamide Riboside Triacetate Chloride.

In one embodiment, a substantially crystalline Nicotinamide Riboside Triacetate compound is described, or a salt, or solvate thereof, having a chemical purity of greater than about 90% (w/w) and containing less than about 5000 ppm ethanol. In a further embodiment, the substantially crystalline Nicotinamide Riboside Triacetate compound is Nicotinamide Riboside Triacetate Chloride in substantially a beta anomer form.

In another embodiment, a method is described for making a Nicotinamide Riboside Triacetate compound, or a salt, or solvate thereof, including the steps of: (a) adding a mass of Crude Nicotinamide Riboside Triacetate to a volume of a first solvent to form a reaction mixture; (b) heating the reaction mixture to a temperature of about 20° C. to about 60° C.; (c) cooling the reaction mixture; (d) adding a second solvent; and (e) isolating the substantially crystalline compound Nicotinamide Riboside Triacetate, or a salt, or a solvate thereof as a crystalline powder. Optionally, the method may include step (c1) seeding the reaction mixture with crystalline compound Nicotinamide Riboside Triacetate, or a salt, or a solvate thereof after step (c)

### BRIEF DESCRIPTION OF THE DRAWINGS

FIG. 1 is a schematic of the synthetic sequence used to produce Crystalline Beta Nicotinamide Riboside Triacetate Chloride

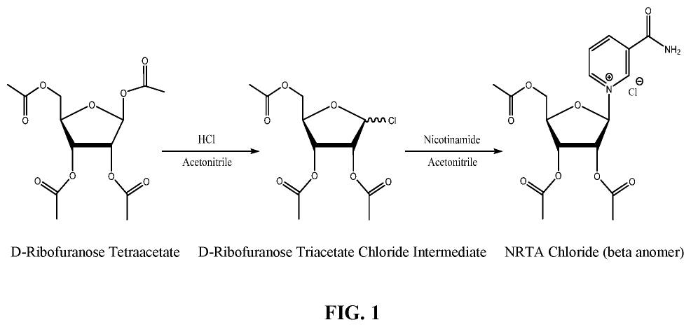

### DETAILED DESCRIPTION

Nicotinamide riboside (“NR”) is a pyridinium compound having the formula (I):

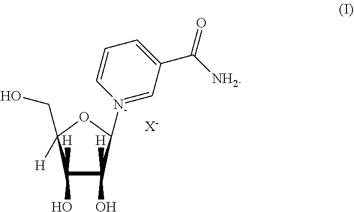

NR of formula (I) can include salts or solvates. Salts may include counterions (defined as “X⁻”) selected from chloride, bromide, iodide, and the like. For example, one useful salt is the chloride salt of NR (“NR—Cl”). Further salts may include, but are not limited to, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, trifluoroacetate, glycoloate, glucuronate, pyruvate, anthranilate, 4-hydroxybenzoate, phenylacetate, mandelate, pamoate, methanesulfonate, ethanesulfonate, benzenesulfonate, pantothenate, 2-hydroxyethanesulfonate, p-toluenesulfonate, sulfanilate, cyclohexylaminesulfonate, alginate, beta-hydroxybutyrate, salicylate, galactarate, galacturonate, and the like. For NAR, NAMN and NMN, and the like, optionally wherein when X⁻ is absent, optionally the counterion is an internal salt.

NR is hydrophilic, although susceptible to hydrolysis. This presents a unique requirement such that chemical stability requires microencapsulation of a water soluble compound. This is a reversal of the common formulator's technique to microencapsulate a hydrophobic, lipophilic, or water-insoluble material in order to provide better bioavailability.

In a further aspect, derivatives of NR are contemplated having the formula (Ia) or a salt, solvate, or prodrug thereof:

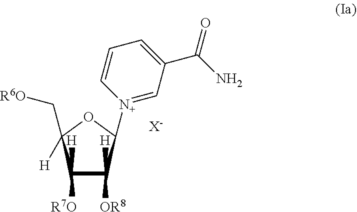

wherein R⁶ is selected from the group consisting of hydrogen, —C(O)R′, —C(O)OR′, C(O)NHR′, substituted or unsubstituted (C₁-C₂₄)alkyl, substituted or unsubstituted (C₃-C₈)cycloalkyl, substituted or unsubstituted aryl, substituted or unsubstituted heteroaryl, and substituted or unsubstituted heterocycle;
R′ is selected from the group consisting of hydrogen, —(C₁-C₂₄)alkyl, —(C₃-C₈)cycloalkyl, aryl, heteroaryl, heterocycle, aryl(C₁-C₂₄)alkyl, and heterocycle(C₁-C₂₄)alkyl; and
R⁷ and R⁸ are independently selected from the group consisting of hydrogen, —C(O)R′, —C(O)OR′, —C(O)NHR′, substituted or unsubstituted (C₁-C₂₄)alkyl, substituted or unsubstituted (C₃-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl.

This disclosure also includes other NAD+ precursors, such as, but not limited to, one or more nicotinyl riboside compounds selected from nicotinic acid riboside (NAR, II), nicotinamide mononucleotide (NMN, III), nicotinic acid mononucleotide (NaMN, IV), reduced nicotinamide riboside (NRH, V), reduced nicotinic acid riboside (NARH, VI), NR triacetate (NRTA, VII which is a species of Ia), NAR triacetate (NARTA, VIII), NRH triacetate (NRH-TA, IX), or NARH triacetate (NARH-TA, X), and salts, solvates, or mixtures thereof, or derivatives thereof.

Nicotinic acid riboside (NAR) is a pyridinium nicotinyl compound having the formula (II):

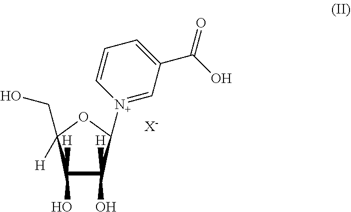

and optionally where X− is absent, NAR is an inner salt (zwitterionic species).

Nicotinamide mononucleotide (NMN) is a pyridinium nicotinyl compound having the formula (III):

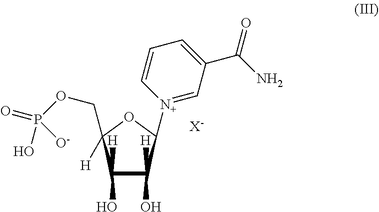

and optionally where X− is absent, NMN can be an inner salt.

Nicotinic acid mononucleotide (NaMN) is a pyridinium nicotinyl compound having the formula (IV):

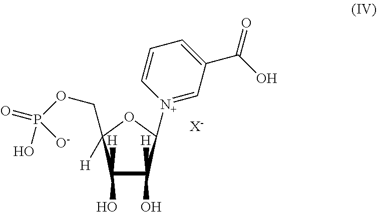

and optionally where X− is absent, NaMN can be an inner salt.

Salts may include counterions (defined as “X⁻”) selected from chloride, bromide, iodide, and the like, or alternatively, organic counterions as shown in Formula (I). For example, one useful salt is the chloride salt of NR (“NR—Cl”). Further salts including phosphate salts which may include, but are not limited to one or more of sodium, potassium, lithium, magnesium, calcium, strontium, or barium. Reduced nicotinamide riboside (“NRH”) is a 1,4-dihydropyridyl reduced nicotinyl compound having the formula (V):

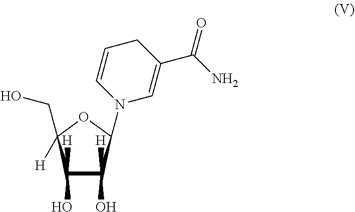

Reduced nicotinic acid riboside (“NARH”) is a 1,4-dihydropyridyl reduced nicotinyl compound having the formula (VI):

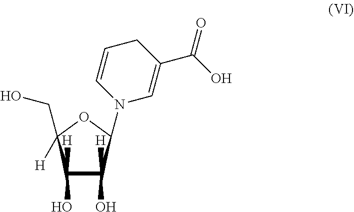

In certain species of compound (Ia), the free hydrogens of hydroxyl groups on the ribose moiety of nicotinamide riboside (NR, I) can be substituted with acetyl groups (CH₃—C(═O)—) to form 1-(2′,3′,5′-triacetyl-beta-D-ribofuranosyl)-nicotinamide (“NR triacetate” or “NRTA”) having the formula (VII):

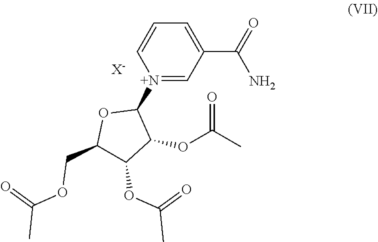

where X⁻ is defined as above.

The free hydrogens of hydroxyl groups on the ribose moiety of nicotinic acid riboside (NAR, II) can be substituted with acetyl groups (CH₃—C(═O)—) to form 1-(2′,3′,5′-triacetyl-beta-D-ribofuranosyl)-nicotinic acid (“NAR triacetate” or “NARTA”) having the formula (VIII):

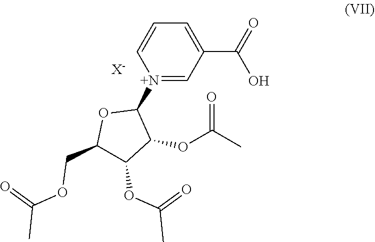

and optionally where X− is absent, NARTA is an inner salt.

The free hydrogens of hydroxyl groups on the ribose moiety of reduced nicotinamide riboside (NRH, V) can be substituted with acetyl groups (CH₃—C(═O)—) to form 1-(2′,3′,5′-triacetyl-beta-D-ribofuranosyl)-1,4-dihydronicotinamide (“NRH triacetate” or “NRH-TA”) having the formula (IX):

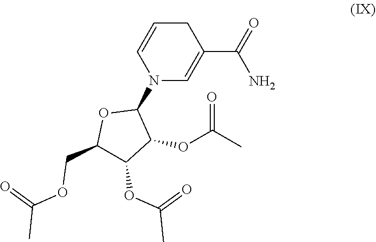 [SMILES: CC(=O)OC[C@H]1O[C@@H](N2C=CCC(C(N)=O)=C2)[C@H](OC(C)=O)[C@@H]1OC(C)=O]

The free hydrogens of hydroxyl groups on the ribose moiety of reduced nicotinic acid riboside (NARH, VI) can be substituted with acetyl groups (CH₃—C(═O)—) to form 1-(2′,3′,5′-triacetyl-beta-D-ribofuranosyl)-1,4-dihydronicotinic acid (“NARH triacetate” or “NARH-TA”) having the formula (X):

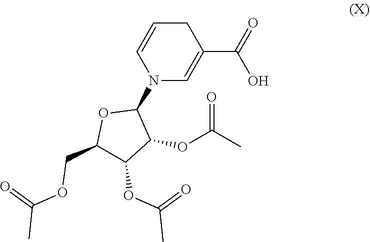 [SMILES: CC(=O)OC[C@H]1O[C@@H](N2C=CCC(C(=O)O)=C2)[C@H](OC(C)=O)[C@@H]1OC(C)=O]

For each of nicotinamide riboside (NR, I), nicotinic acid riboside (NAR, II), nicotinamide mononucleotide (NMN, III), nicotinic acid mononucleotide (NaMN, IV), reduced nicotinamide riboside (NRH, V), reduced nicotinic acid riboside (NARH, VI), nicotinamide riboside triacetate (NRTA, VII), nicotinic acid riboside triacetate (NARTA, VIII), reduced nicotinamide riboside triacetate (NRH-TA, IX), and reduced nicotinic acid riboside triacetate (NARH-TA, X), optionally X⁻ as counterion is absent, or when X⁻ is present, X⁻ is selected from the group consisting of fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, sulfate, tartrate, hydrogen tartrate, malate, hydrogen malate, maleate, fumarate, citrate, stearate, palmitate, myristate, laurate, caprate, caprylate, caproate, oleate, linoleate, sulfonate, trifluoromethanesulfonate, trichloromethanesulfonate, tribromomethanesulfonate, trichloroacetate, tribromoacetate, trifluoroacetate, glycoloate, glucuronate, pyruvate, anthranilate, 4-hydroxybenzoate, phenylacetate, mandelate, pamoate, methanesulfonate, ethanesulfonate, benzenesulfonate, pantothenate, 2-hydroxyethanesulfonate, p-toluenesulfonate, sulfanilate, cyclohexylaminesulfonate, alginate, beta-hydroxybutyrate, salicylate, galactarate, galacturonate, and the like; and,
optionally wherein when X⁻ is absent, optionally the counterion is an internal salt;
optionally X⁻ is an anion of a substituted or unsubstituted carboxylic acid selected from monocarboxylic acid, a dicarboxylic acid, or a polycarboxylic acid;
optionally X⁻ is an anion of a substituted monocarboxylic acid, further optionally an anion of a substituted propanoic acid (propanoate or propionate), or an anion of a substituted acetic acid (acetate), or an anion of a hydroxyl-propanoic acid, or an anion of 2-hydroxypropanoic acid (being lactic acid; the anion of lactic acid being lactate), or a trihaloacetate selected from trichloroacetate, tribromoacetate, or trifluoroacetate; and,
optionally X⁻ is an anion of an unsubstituted monocarboxylic acid selected from formic acid, acetic acid, propionic acid, or butyric acid, or an anion of a long chain fatty acid including saturated, unsaturated and polyunsaturated fatty acids with carbon chain lengths of C₆-C₂₄ (such as, for example, stearic acid, palmitic acid, myristic acid, lauric acid, capric acid, caprylic acid, caproic acid, oleic acid, linoleic acid, omega-6 fatty acid, omega-3 fatty acid); the anions being formate, acetate, propionate, butyrate, and stearate, and the like, respectively; and,
optionally X⁻ is an anion of a substituted or unsubstituted amino acid, i.e., amino-monocarboxylic acid or an amino-dicarboxylic acid, optionally selected from glutamic acid and aspartic acid, the anions being glutamate and aspartate, respectively; or, alternatively, selected from alanine, beta-alanine, arginine, asparagine, cysteine, glutamine, glycine, histidine, isoleucine, leucine, lysine, methionine, phenylalanine, proline, serine, threonine, tryptophan, or tyrosine, and,
optionally X⁻ is an anion of ascorbic acid, being ascorbate; and,
optionally X⁻ is a halide selected from fluoride, chloride, bromide, or iodide; and,
optionally X⁻ is an anion of a substituted or unsubstituted sulfonate, further optionally a trihalomethanesulfonate selected from trifluoromethanesulfonate, tribromomethanesulfonate, or trichloromethanesulfonate; and
optionally X⁻ is an anion of a substituted or unsubstituted carbonate, further optionally hydrogen carbonate.

In yet another embodiment, the present disclosure relates to crystalline forms of nicotinic acid riboside (1-(beta-D-ribofuranosyl)-nicotinic acid, NAR), including, but not limited to, a “Form II” or a “Form I” of nicotinic acid riboside (NAR), and methods of preparation thereof, as disclosed in U.S. Pat. Nos. 11,214,589 and 9,975,915, respectively.

In yet another embodiment, the present disclosure relates to crystalline forms of nicotinamide riboside triacetate chloride (NRTA-C1) form I, and methods of preparation thereof, as disclosed in U.S. Pat. No. 9,975,915.

In yet another embodiment, the present disclosure relates to crystalline forms of nicotinic acid riboside triacetate (1-(2′,3′,5′-triacetyl-beta-D-ribofuranosyl)-nicotinic acid, “NAR triacetate,” or “NARTA”), including, but not limited to, a “Form II” or a “Form I” of nicotinic acid riboside triacetate (NARTA), and methods of preparation thereof as disclosed in U.S. Pat. Nos. 11,214,589 and 10,689,411, respectively.

Crystalline forms, a.k.a. polymorphic crystal forms or “polymorphs,” of useful molecules can have advantageous properties relative to the respective amorphous forms of such molecules. For example, crystal forms are often easier to handle and process, for example, when preparing compositions that include the crystal forms. Crystalline forms typically have greater storage stability and are more amenable to purification. The use of a crystalline form of a pharmaceutically useful compound can also improve the performance characteristics of a pharmaceutical product that includes the compound. Obtaining the crystalline form also serves to enlarge the repertoire of materials that formulation scientists have available for formulation optimization, for example by providing a product with different properties, e.g., better processing or handling characteristics, improved dissolution profile, or improved shelf-life. The flow of powders is critical in formulation development for making tablets and capsules. The tableting process is based on powder volume and the flow of the powder to maintain tablet weight uniformity. Therefore, designing the process and having consistent control over the flow properties of the powder are critical in achieving optimized production. The development of the crystallization process resulting in a form with novel enhanced physical and/or stability properties allows for formulation advancement compared to the physically inferior properties exhibited by other forms.

### Definitions

As used herein, the term “solvent” refers to a compound or mixture of compounds including, but not limited to, water, water in which an ionic compound has been dissolved, acetic acid, acetone, acetonitrile, benzene, 1-butanol, 2-butanol, t-butyl alcohol (“TBA”, “t-BuOH”), 2-butanone, carbon tetrachloride, chlorobenzene, chloroform, cyclohexane, 1,2-dichloroethane (“DCE”), diethylene glycol, diethyl ether (“Et₂O”), diglyme (diethylene glycol dimethyl ether), 1,2-dimethoxyethane (“DME”), N,N-dimethylformamide (“DMF”), dimethylsulfoxide (“DMSO”), 1,4-dioxane, ethanol, ethyl acetate (“EtOAc”), ethylene glycol, glycerin, heptanes, hexamethylphosphoramide (“HMPA”), hexamethylphosphorus triamide (“HMPT”), hexane, methanol (“MeOH”), methyl t-butyl ether (“MTBE”), methylene chloride (“DCM,” “CH₂Cl₂”), N-methyl-2-pyrrolidinone (“NMP”), nitromethane, pentane, petroleum ether, 1-propanol (“n-propanol,” “n-PrOH”), 2-propanol (“isopropanol,” “iPrOH”), pyridine, tetrahydrofuran (“THF”), toluene, triethylamine (“TEA,” “Et₃N”), o-xylene, m-xylene, and/or p-xylene, and the like. Solvent classes may include hydrocarbon, aromatic, aproptic, polar, alcoholic and mixtures thereof.

According to particular embodiments, the compounds or derivatives prepared according to embodiments of the methods of the present disclosure can comprise compounds or derivatives, or salts, hydrates, solvates, or prodrugs thereof, or crystalline forms thereof, substantially free of solvents or other by-products, generally, or free of a particular solvent or by-product. In certain embodiments, by “substantially free” is meant greater than about 80% by weight free of solvents or by-products, or greater than about 80% by weight free of a particular solvent or by-product, more preferably greater than about 90% by weight free of solvents or by-products, or greater than about 90% by weight free of a particular solvent or by-product, even more preferably greater than about 95% by weight free of solvents or by-products, or greater than about 95% by weight free of a particular solvent or by-product, even more preferably greater than 98% by weight free of solvents or by-products, or greater than about 98% by weight free of a particular solvent or by-product, even more preferably greater than about 99% by weight free of solvents or by-products, or greater than about 99% by weight free of a particular solvent or by-product, even more preferably greater than about 99.99% by weight free of solvents or by-products, or greater than about 99.99% by weight free of a particular solvent or by-product, and most preferably quantitatively free of solvents or by-products, or quantitatively free of a particular solvent or by-product.

For preparing pharmaceutical compositions from a crystalline form of Nicotinamide Riboside chloride or a hydrate, solvate, or prodrug thereof, prepared according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active components. In tablets, the active component is mixed with the carrier having the necessary binding capacity in suitable proportions and compacted in the shape and size desired.

The powders and tablets preferably contain from about five or ten to about seventy percent of the active crystalline form of Nicotinamide Riboside (NR) or Nicotinamide Riboside triacetate (NRTA, VII), or a salt, a hydrate, a solvate, or a prodrug thereof, for example a chloride salt (NRTA-Cl), or mixtures thereof, prepared according to the methods of the present disclosure. Suitable carriers are microcrystalline cellulose, sugar, lactose, pectin, dextrin, starch, gelatin, tragacanth, methylcellulose, sodium carboxymethylcellulose, a low melting wax, cocoa butter, and the like, and other excipients may include magnesium stearate, stearic acid, talc, silicon dioxide, etc. Dosages of the active form of nicotinamide riboside (NR), or Nicotinamide Riboside triacetate (NRTA, VII), or a salt, a hydrate, a solvate, or a prodrug thereof, for example a chloride salt (NRTA-Cl), or mixtures thereof, may be between about 10 mg to about 10000 mg in the preparation for example. The term “preparation” is intended to include the formulation of active compound with encapsulating material as carrier providing a capsule in which the active component, with or without carriers, is surrounded by a carrier, which is thus in association with it. Tablets, powders, capsules, pills, sachets, and lozenges are included. Tablets, powders, capsules, pills, sachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The crystalline forms of nicotinamide riboside (NR), or Nicotinamide Riboside triacetate (NRTA, VII), or a salt, a hydrate, a solvate, or a prodrug thereof, for example a chloride salt (NRTA-Cl), or mixtures thereof prepared according to the methods of the present disclosure may thus be formulated for parenteral administration (e.g., by injection, for example bolus injection or continuous infusion) and may be presented in unit dose for example in ampoules, pre-filled syringes, small volume infusion, or in multi-dose containers with an added preservative). The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for reconstitution with a suitable vehicle, e.g., sterile, pyrogen-free water, before use.

The powders and tablets preferably contain from about 1 to about 99.99 percent of the active crystalline form of nicotinamide riboside (NR, I) or nicotinamide riboside triacetate (NRTA, VII), or salt, hydrate, solvate, or prodrug thereof, prepared according to the methods of the present disclosure. Suitable carriers are microcrystalline cellulose, sugar, lactose, pectin, dextrin, starch, gelatin, tragacanth, methylcellulose, sodium carboxymethylcellulose, a low melting wax, cocoa butter, and the like, and other excipients may include magnesium stearate, stearic acid, talc, silicon dioxide, etc. Dosages of the active form of nicotinamide riboside (NR, I) or nicotinamide riboside triacetate (NRTA, VII) may be between about 10 mg to about 10000 mg in the preparation for example. The term “preparation” is intended to include the formulation of active compound with encapsulating material as carrier providing a capsule in which the active component, with or without carriers, is surrounded by a carrier, which is thus in association with it. Tablets, powders, capsules, pills, sachets, and lozenges are included. Tablets, powders, capsules, pills, sachets, and lozenges can be used as solid forms suitable for oral administration. Liquid preparations include solutions, suspensions, and emulsions, for example, water or water-propylene glycol solutions.

For example, parenteral injection liquid preparations can be formulated as solutions in aqueous polyethylene glycol solution. The crystalline forms of nicotinamide riboside (NR, I) or nicotinamide riboside triacetate (NRTA, VII), or salts, hydrates, solvates, or prodrugs thereof prepared according to the methods of the present disclosure may thus be formulated for parenteral administration (e.g., by injection, for example bolus injection or continuous infusion) and may be presented in unit dose for example in ampoules, pre-filled syringes, small volume infusion, or in multi-dose containers with an added preservative). The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use. The method of administration may be via inhalation and topical routes. Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth include lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example with a dropper, pipette, or spray. The compositions may be provided in single or multi-dose form. In compositions intended for administration to the respiratory tract, including intranasal compositions, the compound or derivative will generally have a small particle size, for example on the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred compositions.

Aqueous solutions suitable for oral use can be prepared by dis solving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

The crystalline forms of Beta-Nicotinamide Riboside Triacetate that are prepared by the methods of the present disclosure may take the form of salts. The term “salts” embraces addition salts of free acids or free bases that are crystalline forms of Beta-Nicotinamide Riboside Triacetate that are prepared by the methods of the present disclosure. The term “pharmaceutically acceptable salt” refers to salts that possess toxicity profiles within a range that affords utility in pharmaceutical applications.

Further details on techniques for formulation may be found in the latest edition of Remington's Pharmaceutical Sciences (Mack Publishing Co., Easton, PA).

Additionally, the embodiments of the present methods for treating and/or preventing symptoms, diseases, disorders, or conditions associated with, or having etiologies involving, vitamin B3 deficiency and/or that would benefit from increased mitochondrial activity in a mammalian subject address limitations of existing technologies to treat or prevent symptoms, diseases, disorders, or conditions associated with, or having etiologies involving, vitamin B3 deficiency and/or that would benefit from increased mitochondrial activity.

In certain embodiments, the present invention provides methods for treating and/or preventing symptoms, diseases, disorders, or conditions associated with, or having etiologies involving, vitamin B3 deficiency. Exemplary symptoms, diseases, disorders, or conditions associated with, or having etiologies involving, vitamin B3 deficiency that may be treated and/or prevented in accordance with the methods described include indigestion, fatigue, canker sores, vomiting, poor circulation, burning in the mouth, swollen red tongue, and depression. Severe vitamin B3 deficiency can cause a condition known as pellagra, a premature aging condition that is characterized by cracked, scaly skin, dementia, and diarrhea. Other conditions characterized by premature or accelerated aging include Cockayne Syndrome, Neill-Dingwall Syndrome, progeria, and the like.

In certain embodiments, the present invention provides methods for treating and/or preventing symptoms, diseases, disorders, or conditions that would benefit from increased mitochondrial activity. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In certain embodiments, symptoms, diseases, disorders, or conditions that would benefit from increased mitochondrial activity include symptoms, diseases, disorders, or conditions associated with mitochondrial dysfunction.

In certain embodiments, methods for treating and/or preventing symptoms, diseases, disorders, or conditions that would benefit from increased mitochondrial activity may comprise identifying a subject suffering from a mitochondrial dysfunction. Methods for diagnosing a mitochondrial dysfunction that may involve molecular genetic, pathologic, and/or biochemical analysis are summarized in Bruce H. Cohen & Deborah R. Gold, Mitochondrial cytopathy in adults: what we know so far, 68 CLEVELAND CLINIC J. MED. 625 (2001). One method for diagnosing a mitochondrial dysfunction is the Thor-Byrneier scale (see, e.g., Cohen & Gold 2001; S. Collins et al., Respiratory Chain Encephalomyopathies: A Diagnostic Classification, 36 EUROPEAN NEUROLOGY 260 (1996)).

Mitochondria are critical for the survival and proper function of almost all types of eukaryotic cells. Mitochondria in virtually any cell type can have congenital or acquired defects that affect their function. Thus, the clinically significant signs and symptoms of mitochondrial defects affecting respiratory chain function are heterogeneous and variable depending on the distribution of defective mitochondria among cells and the severity of their deficits, and upon physiological demands upon the affected cells. Nondividing tissues with high energy requirements, e.g., nervous tissue, skeletal muscle, and cardiac muscle are particularly susceptible to mitochondrial respiratory chain dysfunction, but any organ system can be affected.

Symptoms, diseases, disorders, and conditions associated with mitochondrial dysfunction include symptoms, diseases, disorders, and conditions in which deficits in mitochondrial respiratory chain activity contribute to the development of pathophysiology of such symptoms, diseases, disorders, or conditions in a mammal. This includes 1) congenital genetic deficiencies in activity of one or more components of the mitochondrial respiratory chain, wherein such deficiencies are caused by a) oxidative damage during aging; b) elevated intracellular calcium; c) exposure of affected cells to nitric oxide; d) hypoxia or ischemia; e) microtubule-associated deficits in axonal transport of mitochondria; or f) expression of mitochondrial uncoupling proteins.

Symptoms, diseases, disorders, or conditions that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis. Exemplary symptoms, diseases, disorders, or conditions that would benefit from increased mitochondrial activity include, for example, AD (Alzheimer's Disease), ADPD (Alzheimer's Disease and Parkinson's Disease), AMDF (Ataxia, Myoclonus and Deafness), auto-immune disease, lupus, lupus erythematosus, SLE (systemic lupus erythematosus), cataracts, cancer, CIPO (Chronic Intestinal Pseudoobstruction with myopathy and Ophthalmoplegia), congenital muscular dystrophy, CPEO (Chronic Progressive External Ophthalmoplegia), DEAF (Maternally inherited DEAFness or aminoglycoside-induced DEAFness), DEMCHO (Dementia and Chorea), diabetes mellitus (Type I or Type II), DID-MOAD (Diabetes Insipidus, Diabetes Mellitus, Optic Atrophy, Deafness), DMDF (Diabetes Mellitus and Deafness), dystonia, Exercise Intolerance, ESOC (Epilepsy, Strokes, Optic atrophy, and Cognitive decline), FBSN (Familial Bilateral Striatal Necrosis), FICP (Fatal Infantile Cardiomyopathy Plus, a MELAS -associated cardiomyopathy), GER (Gastrointestinal Reflux), HD (Huntington's Disease), KSS (Kearns Sayre Syndrome), “later-onset” myopathy, LDYT (Leber's hereditary optic neuropathy and DYsTonia), Leigh's Syndrome, LHON (Leber Hereditary Optic Neuropathy), LIMM (Lethal Infantile Mitochondrial Myopathy), MDM (Myopathy and Diabetes Mellitus), MELAS (Mitochondrial Encephalomyopathy, Lactic Acidosis, and Stroke-like episodes), MEPR (Myoclonic Epilepsy and Psychomotor Regression), MERME (MERRF/MELAS overlap disease), MERRF (Myoclonic Epilepsy and Ragged Red Muscle Fibers), MHCM (Maternally Inherited Hypertrophic CardioMyopathy), MICM (Maternally Inherited Cardiomyopathy), MILS (Maternally Inherited Leigh Syndrome), Mitochondrial Encephalocardiomyopathy, Mitochondrial Encephalomyopathy, MM (Mitochondrial Myopathy), MMC (Maternal Myopathy and Cardiomyopathy), MNGIE (Myopathy and external ophthalmoplegia, Neuropathy, Gastro -Intestinal, Encephalopathy), Multisystem Mitochondrial Disorder (myopathy, encephalopathy, blindness, hearing loss, peripheral neuropathy), NARP (Neurogenic muscle weakness, Ataxia, and Retinitis Pigmentosa; alternate phenotype at this locus is reported as Leigh Disease), PD (Parkinson's Disease), Pearson's Syndrome, PEM (Progressive Encephalopathy), PEO (Progressive External Ophthalmoplegia), PME (Progressive Myoclonus Epilepsy), PMPS (Pearson Marrow-Pancreas Syndrome), psoriasis, RTT (Rett Syndrome), schizophrenia, SIDS (Sudden Infant Death Syndrome), SNHL (Sensorineural Hearing Loss), Varied Familial Presentation (clinical manifestations range from spastic paraparesis to multisystem progressive disorder & fatal cardiomyopathy to truncal ataxia, dysarthria, severe hearing loss, mental regression, ptosis, ophthalmoparesis, distal cyclones, and diabetes mellitus), or Wolfram syndrome.

Other symptoms, diseases, disorders, and conditions that would benefit from increased mitochondrial activity include, for example, Friedreich's ataxia and other ataxias, amyotrophic lateral sclerosis (ALS) and other motor neuron diseases, macular degeneration, epilepsy, Alpers syndrome, Multiple mitochondrial DNA deletion syndrome, MtDNA depletion syndrome, Complex I deficiency, Complex II (SDH) deficiency, Complex III deficiency, Cytochrome c oxidase (COX, Complex IV) deficiency, Complex V deficiency, Adenine Nucleotide Translocator (ANT) deficiency, Pyruvate dehydrogenase (PDH) deficiency, Ethylmalonic aciduria with lactic acidemia, Refractory epilepsy with declines during infection, Asperger syndrome with declines during infection, Autism with declines during infection, Attention deficit hyperactivity disorder (ADHD), Cerebral palsy with declines during infection, Dyslexia with declines during infection, materially inherited thrombocytopenia and leukemia syndrome, MARIAHS syndrome (Mitochondrial ataxia, recurrent infections, aphasia, hypouricemia/hypomyelination, seizures, and dicarboxylic aciduria), ND6 dystonia, Cyclic vomiting syndrome with declines during infection, 3-Hydroxy isobutyric aciduria with lactic acidemia, Diabetes mellitus with lactic acidemia, Uridine responsive neurologic syndrome (URNS), Dilated cardiomyopathy, Splenic Lymphoma, or Renal Tubular Acidosis/Diabetes/Ataxis syndrome.

In other embodiments, the present invention provides methods for treating a mammal (e.g., human) suffering from mitochondrial disorders arising from, but not limited to, Post-traumatic head injury and cerebral edema, Stroke (invention methods useful for treating or preventing reperfusion injury), Lewy body dementia, Hepatorenal syndrome, Acute liver failure, NASH (non-alcoholic steatohepatitis), Anti-metastasis/prodifferentiation therapy of cancer, Idiopathic congestive heart failure, Atrial fibrillation (non-valvular), Wolff-Parkinson-White Syndrome, Idiopathic heart block, Prevention of reperfusion injury in acute myocardial infarctions, Familial migraines, Irritable bowel syndrome, Secondary prevention of non-Q wave myocardial infarctions, Premenstrual syndrome, Prevention of renal failure in hepatorenal syndrome, Anti-phospholipid antibody syndrome, Eclampsia/pre-eclampsia, Oopause infertility, Ischemic heart disease/Angina, and Shy-Drager and unclassified dysautonomia syndromes.

In still another embodiment, there are provided methods for the treatment of mitochondrial disorders associated with pharmacological drug-related side effects. Types of pharmaceutical agents that are associated with mitochondrial disorders include reverse transcriptase inhibitors, protease inhibitors, inhibitors of DHOD, and the like. Examples of reverse transcriptase inhibitors include, for example, Azidothymidine (AZT), Stavudine (D4T), Zalcitabine (ddC), Didanosine (DDI), Fluoroiodoarauracil (FIAU), Lamivudine (3TC), Abacavir, and the like. Examples of protease inhibitors include, for example, Ritonavir, Indinavir, Saquinavir, Nelfinavir, and the like. Examples of inhibitors of dihydroorotate dehydrogenase (DHOD) include, for example, Leflunomide, Brequinar, and the like.

Reverse transcriptase inhibitors not only inhibit reverse transcriptase but also polymerase gamma, which is required for mitochondrial function. Inhibition of polymerase gamma activity (e.g., with a reverse transcriptase inhibitor) therefore leads to mitochondrial dysfunction and/or a reduced mitochondrial mass, which manifests itself in patients as hyperlactatemia. This type of condition may benefit from an increase in the number of mitochondria and/or an improvement in mitochondrial function.

Common symptoms of mitochondrial diseases include cardiomyopathy, muscle weakness and atrophy, developmental delays (involving motor, language, cognitive, or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

### Embodiments of the Current Invention

The referenced invention provides a scalable crystallization process that produces crystalline Beta-Nicotinamide Riboside Triacetate Chloride. The improved process characteristics described herein generate crystalline material with low residual solvent content, large crystal particle size, narrow particle size distribution, and high yields suitable for use as a commercial dietary supplement.

Novel components of the invention include: improved crystal size and particle size distribution versus alternative crystallization processes; low residual solvent content versus crystalline material generated via alternative crystallization processes; and improved yield versus alternative crystallization processes. The process described herein above effects a preparation of the above crystalline beta Nicotinamide Riboside Triacetate Chloride.

In one embodiment, a method of making a crystalline Nicotinamide Riboside Triacetate Chloride can include the steps as disclosed in U.S. Pat. No. 9,975,915, herein incorporated by reference in its entirety.

In one embodiment, a method of making a crystalline Nicotinamide Riboside Triacetate Chloride can include the steps of:
a) adding a mass of Crude Nicotinamide Riboside Triacetate Chloride to a vessel at a first temperature, optionally the mass of Crude Nicotinamide Riboside Triacetate Chloride is between about 65 g and about 80 g, and the first temperature is between about 18° C. and about 23° C.;
b) adding a mass of water and mass of ethanol to create a reaction mixture, optionally the mass of water is between about 75 g and about 90 g and the mass of ethanol is between about 38 g and about 50 g;
c) stirring the reaction mixture and heating a second temperature, optionally the second temperature is between about 48° C. and about 52° C.;
d) once at the second temperature, cooling the vessel to a third temperature, optionally the third temperature is between about 28° C. and about 32° C.;
e) slowly metering the vessel with a mass of ethanol at a first rate, optionally the mass of ethanol is between about 850 g about 950 g, and the first rate is between about 8mL/min and about 12 mL/min;
f) holding the vessel at the third temperature for a first time period, optionally the first time period is between about 45 minutes and about 75 minutes;
g) cooling the vessel to a fourth temperature, optionally the fourth temperature is between about −8° C. and about −12° C. and holding the fourth temperature to a second period of time, optionally the second period of time is between about 8 hours and about 16 hours;
h) observing crystal formation at a fifth temperature or before the fifth temperature, optionally, the fifth temperature is between about −5° C. and about −9° C.; and
i) obtaining Nicotinamide Riboside Triacetate Chloride as a white, crystalline powder.

The crystalline forms of Nicotinamide Riboside Triacetate Chloride of the present disclosure may be isolated from their reaction mixtures and purified by standard techniques such as filtration, liquid-liquid extraction, solid phase extraction, distillation, recrystallization, or chromatography, including flash column chromatography, preparative TLC, HPTLC, HPLC, or rp-HPLC. One preferred method for preparation of the crystalline forms of Nicotinamide Riboside Triacetate Chloride of the present disclosure, comprises crystallizing the compound, or salt, hydrate, solvate, or prodrug thereof, from a solvent, to form, preferably, a crystalline form of the compound or derivative, or salt, hydrate, solvate, or prodrug thereof. Following crystallization, the crystallization solvent is removed by a process other than evaporation, for example, filtration or decanting, and the crystals are then preferably washed using pure solvent (or a mixture of pure solvents). Preferred solvents for crystallization include water; alcohols, particularly alcohols containing up to four carbon atoms, such as methanol, ethanol, isopropanol, butan-1-ol, butan-2-ol, and 2-methyl-2-propanol; ethers, for example diethyl ether, diisopropyl ether, t-butyl methyl ether, 1,2-dimethoxyethane, tetrahydrofuran, and 1,4-dioxane; carboxylic acids, for example formic acid and acetic acid; hydrocarbon solvents, for example pentane, hexane, and toluene; and mixtures thereof, particularly aqueous mixtures such as aqueous methanol, ethanol, isopropanol, and acetone. Pure solvents, preferably at least analytical grade, and more preferably pharmaceutical grade are preferably used. In preferred embodiments of the methods of the invention, the crystalline forms are so isolated. As described above, solvates of the crystalline NRTA chloride may include one or more of the solvents listed above.

In one embodiment, a method of making a crystalline Nicotinamide Riboside Triacetate Chloride can include the steps of:
a) adding a mass of Crude Nicotinamide Riboside Triacetate Chloride to a vessel at a first temperature, optionally the mass of Crude Nicotinamide Riboside Triacetate Chloride is between about 65 g and about 80 g, and the first temperature is between about 18° C. and about 23° C.;
b) adding a mass of water and mass of ethanol to create a reaction mixture, optionally the mass of water is between about 75 g and about 90 g and the mass of ethanol is between about 38 g and about 50 g;
c) stirring the reaction mixture and heating a second temperature, optionally the second temperature is between about 28° C. and about 32° C.;
d) slowly metering the vessel with a mass of ethanol at a first rate, optionally the mass of ethanol is between about 850 g about 950 g, and the first rate is between about 8 mL/min and about 12 mL/min;
e) cooling the vessel to a third temperature, optionally the third temperature is between about −8° C. and about −12° C. and holding the fourth temperature to a first period of time, optionally the first period of time is between about 8 hours and about 16 hours;
f) observing crystal formation at a fourth temperature or before the fourth temperature, optionally, the fourth temperature is between about −5° C. and about −9° C.; and
g) obtaining Nicotinamide Riboside Triacetate Chloride as a white, crystalline powder.

In one embodiment, a method of making a crystalline Nicotinamide Riboside Triacetate Chloride can include the steps of:
a) adding a mass of Crude Nicotinamide Riboside Triacetate Chloride to a vessel at a first temperature, optionally the mass of Crude Nicotinamide Riboside Triacetate Chloride is between about 650 g and about 550 g, and the first temperature is between about 18° C. and about 23° C.;
b) adding a mass of water and mass of ethanol to create a reaction mixture, optionally the mass of water is between about 305 g and about 450 g and the mass of ethanol is between about 150 g and about 250 g;
c) stirring the reaction mixture and heating a second temperature, optionally the second temperature is between about 28° C. and about 32° C.;
d) slowly metering the vessel with a mass of ethanol at a first rate, optionally the mass of ethanol is between about 875 g about 975 g, and the first rate is between about 25 mL/min and about 35 mL/min;
e) cooling the vessel to a third temperature, optionally the third temperature is between about 8° C. and about 12° C.;
f) seeding crystals with a mass of Nicotinamide Riboside Triacetate Chloride, optionally, the mass of Nicotinamide Riboside Triacetate Chloride is between about 4 g and about 10 g;
g) allowing an isotherm for a first period of time, optionally, the first period of time is between about 30 minutes and about 90 minutes;
h) slowly adding a mass of ethanol at second rate, optionally, the mass of ethanol is between about 2100 g and about 2300 g and the second rate is between about 10 mL/min and about 20 mL/min;
i) cooling the vessel to a fourth temperature at a second period of time, optionally, the fourth temperature is between about −5° C. and about 5° C., and the fourth temperature is between about 40 minutes and about 60 minutes; and
j) cooling the vessel to a fifth temperature at a third period of time, optionally, the fifth temperature is between about −15° C. and −25° C., and the third period of time is between about 15 minutes and about 30 minutes; and
obtaining Nicotinamide Riboside Triacetate Chloride as a white, crystalline powder.

In one embodiment, a method of making a crystalline Nicotinamide Riboside Triacetate Chloride can include the steps of:
a) adding a mass of Crude Nicotinamide Riboside Triacetate Chloride to a vessel at a first temperature, optionally the mass of Crude Nicotinamide Riboside Triacetate Chloride is between about 110 g and about 130 g, and the first temperature is between about 18° C. and about 23° C.;
b) adding a mass of water and mass of ethanol to create a reaction mixture, optionally the mass of water is between about 65 g and about 80 g and the mass of ethanol is between about 38 g and about 58 g;
c) stirring the reaction mixture and heating a second temperature, optionally the second temperature is between about 28° C. and about 32° C.;
d) slowly metering the vessel with a mass of ethanol at a first rate, optionally the mass of ethanol is between about 150 g about 190 g, and the first rate is between about 8 mL/min and about 12 mL/min;
e) cooling the vessel to a third temperature, optionally the third temperature is between about 8° C. and about 15° C.
f) seeding crystals by adding a mass of Nicotinamide Riboside Triacetate Chloride, optionally, the mass of Nicotinamide Riboside Triacetate Chloride is between about 1.0 g and 1.5 g;
g) allowing an isotherm for a first period of time, optionally, the first period of time is between about 45 minutes and about 90 minutes;
h) slowly adding a mass of ethanol at a second rate, optionally, the mass of ethanol is between about 400 g and about 475 g, and the second rate is between about 2 mL/min and about 9 mL/min;
g) cooling the vessel to a fourth temperature for a second period of time, optionally, the fourth temperature is between about −5° C. and about 5° C., and the second period of time is between about 225 minutes and about 275 minutes;
i) cooling the vessel to a fifth temperature, for a third period of time, optionally, the fifth temperature is between about −10° C. and about 0° C., and the third period of time is between about 40 minutes and about 60 minutes;
j) cooling the vessel to a sixth temperature, for a fourth period of time, optionally, the sixth temperature is between about −15° C. and about −5° C., and the fourth period of time is between about 10 minutes and about 30 minutes;
k) cooling the vessel to a seventh temperature, for a fifth period of time, optionally, the seventh temperature is between about −15° C. and about −5° and the fifth period of time is between about 8 hours and about 16 hours; and
l) obtaining Nicotinamide Riboside Triacetate Chloride as a white, crystalline powder.

In one embodiment, a method of making a crystalline Nicotinamide Riboside Triacetate Chloride can include the steps of:
a) adding a mass of Crude Nicotinamide Riboside Triacetate Chloride to a vessel at a first temperature, optionally the mass of Crude Nicotinamide Riboside Triacetate Chloride is between about 110 g and about 130 g, and the first temperature is between about 18° C. and about 23° C.;
b) adding a mass of water and mass of ethanol to create a reaction mixture, optionally the mass of water is between about 65 g and about 80 g and the mass of ethanol is between about 38 g and about 58 g;
c) stirring the reaction mixture and heating a second temperature, optionally the second temperature is between about 28° C. and about 32° C.;
d) slowly metering the vessel with a mass of ethanol at a first rate, optionally the mass of ethanol is between about 150 g about 190 g, and the first rate is between about 8 mL/min and about 12 mL/min;
e) cooling the vessel to a third temperature, optionally the third temperature is between about 10° C. and about 20° C.
f) seeding crystals by adding a mass of Nicotinamide Riboside Triacetate Chloride, optionally, the mass of Nicotinamide Riboside Triacetate Chloride is between about 1.0 g and 1.5 g;
g) allowing an isotherm for a first period of time, optionally, the first period of time is between about 45 minutes and about 90 minutes;
h) slowly adding a mass of ethanol at a second rate, optionally, the mass of ethanol is between about 400 g and about 475 g, and the second rate is between about 2 mL/min and about 9 mL/min;
g) cooling the vessel to a fourth temperature for a second period of time, optionally, the fourth temperature is between about −5° C. and about 5° C., and the second period of time is between about 275 minutes and about 350 minutes;
i) cooling the vessel to a fifth temperature, for a third period of time, optionally, the fifth temperature is between about −10° C. and about 0° C., and the third period of time is between about 40 minutes and about 60 minutes;
j) cooling the vessel to a sixth temperature, for a fourth period of time, optionally, the sixth temperature is between about −15° C. and about −10° C., and the fourth period of time is between about 10 minutes and about 30 minutes;
k) cooling the vessel to a seventh temperature, for a fifth period of time, optionally, the seventh temperature is between about −15° C. and about −5° and the fifth period of time is between about 8 hours and about 16 hours; and
l) obtaining Nicotinamide Riboside Triacetate Chloride as a white, crystalline powder.

In one embodiment, the above crystalline Nicotinamide Riboside Triacetate Chloride can be characterized by a particle size distribution including a size greater than 850 μm at 0.07%, a size between 850-425 μm at 13.92%, a size between 425-250 μm at 47.78%, a size between 250-180 μm at 33.86%, a size between 180-150 μm at 0.29%, a size between 150-125 μm at 2.05%, a size between 125-75 μm at 1.16%, a size between 75-0 μm at 0.55%.

In one embodiment, the above crystalline Nicotinamide Riboside Triacetate Chloride can be characterized by a particle size distribution including a size greater than 850 μm at 0.04%, a size between 850-425 μm at 5.53%, a size between 425-250 μm at 39.32%, a size between 250-180 μm at 26.51%, a size between 180-150 μm at 9.43%, a size between 150-125 μm at 9.59%, a size between 125-75 μm at 7.47%, a size between 75-0 μm at 1.61%.

In one embodiment, the above crystalline Nicotinamide Riboside Triacetate Chloride can be characterized by a particle size distribution including a size greater than 850 μm at 2.52%, a size between 850-425 μm at 44.76%, a size between 425-250 μm at 16.86%, a size between 250-180 μm at 11.16%, a size between 180-150 μm at 6.92%, a size between 150-125 μm at 8.14%, a size between 125-75 μm at 7.76%, a size between 75-μm at 1.69%.

In one embodiment, the above crystalline Nicotinamide Riboside Triacetate Chloride can be characterized by a particle size distribution including a size greater than 850 μm at 0.91%, a size between 850-425 μm at 21.57%, a size between 425-250 μm at 22.95%, a size between 250-180 μm at 21.57%, a size between 180-150 μm at 9.46%, a size between 150-125 μm at 10.27%, a size between 125-75 μm at 10.20%, a size between 75-0 μμm at 2.86%.

In one embodiment, the above crystalline Nicotinamide Riboside Triacetate Chloride can be characterized by a particle size distribution including a size greater than 850 μm at 1.14%, a size between 850-425 μm at 36.68%, a size between 425-250 μm at 18.08%, a size between 250-180 μm at 16.77%, a size between 180-150 μm at 9.86%, a size between 150-125 μm at 10.95%, a size between 125-75 μm at 5.77%, a size between 75-0 μm at 0.17%.

The methods described above may be further understood in connection with the following Examples. In addition, the following non-limiting examples are provided to illustrate the invention. The illustrated preparation procedures are applicable to other embodiments of the present invention. The preparation procedures described as general methods describe what is believed will be typically effective to perform the preparation indicated. However, the person skilled in the art will appreciate that it may be necessary to vary the procedures for any given embodiment of the invention, e.g., vary the order or steps and/or the chemical reagents used. Products may be purified by conventional techniques that will vary, for example, according to the physical properties of the crystalline forms prepared according to the methods of the present invention.

### EXAMPLES

The following non-limiting examples are provided to illustrate the invention. However, the person skilled in the art will appreciate that it may be necessary to vary the procedures for any given embodiment of the invention, e.g., vary the order or steps of the methods.

### Example 1: NRTA-CL Synthesis Preparation of Nicotinamide Riboside Triacetate Chloride

Nicotinamide Riboside Triacetate Chloride (NRTA-Cl) may be prepared as disclosed in U.S. Pat. Nos. 9,975,915 and 10,689,411, herein incorporated by reference in its entirety. In an alternative preparation, a 5L jacketed reactor was charged with 1034 g (3.25 mol) of β-D-Ribofuranose 1,2,3,5-tetraacetate and 1392 g of CH₃CN. The mixture was stirred at 20° C. until dissolution. Following dissolution, the reactor was cooled to −10° C. at which point 13 g (0.16 mol, 0.05 Eq.) of Acetyl Chloride was charged. The reactor was further cooled to −15° C. at which point 146 g (4.06 mol, 1.25 Eq.) anhydrous Hydrogen Chloride gas was sparged into the reaction mixture at 1.5 g/min while maintaining an internal temperature at or below −8° C. Following charge, the reaction was left overnight at −15° C. After isotherm at −15° C., 555 g (4.55 mol, 1.40 Eq.) of Nicotinamide was charged into the reactor along with 757 g CH₃CN. The mixture was stirred for 2 hours at −15° C. and them ramped to 20° C. and held overnight. Following isotherm at 20° C., the reaction was cooled to −5° C. at which time 602.39 g (3.25 mol, 1.00 Eq.) Tributylamine was slowly charged into the reactor. The reactor was ramped to 23° C. and stirred for 3 hours. The crude generated material, Nicotinamide-D-Ribofuranose Triacetate Chloride, was washed with 2500 mL CH₃CN and dried in vacuo at 40° C. 694 g dried material (51% yield) of a pale white, crystalline powder was obtained.

Purity as determined by HPLC: 100.70% Nicotinamide Riboside Triacetate Chloride, 0.513% Nicotinamide.

Residual solvents by GC-MS: Non-Detect ppm Ethanol, 3392.553 ppm Acetonitrile.

### Example 2: NRTA-Cl Crystallization, Self Seeded Preparation of Nicotinamide Riboside Triacetate Chloride Crystals

Crude Nicotinamide Riboside Triacetate Chloride product (˜78 g) produced in a manner similar to Example 1 was added to a 1L jacketed reactor set to about 20° C. To the crude product, about 83 g water and about 42 g ethanol was added. The resultant mixture was stirred and heated to about 50° C. to facilitate dissolution. Once at about 50° C., the reactor was cooled to about 30° C. at which point about 907 g of additional ethanol was slowly metered into the reactor at about 10 mL/min. Following ethanol addition, the reactor was held at about 30° C. for one hour. The reactor was then cooled to about −10° C. and held overnight or between about 8 and about 12 hours. In this method, crystal formation was first observed at about −7° C. About 54.5 g dried material (˜70% mass recovery, ˜76% yield adjusted for dry content & starting material purity) of a white, crystalline powder was obtained.

Crystalline Nicotinamide Riboside Triacetate Chloride included a purity determined by HPLC: ˜99.6%. Nicotinamide Riboside Triacetate Chloride included ˜0.222% of Nicotinamide.

Nicotinamide Riboside Triacetate Chloride included residual solvents determined by GC-MS: ˜3392.35 ppm Ethanol, Non-Detect Acetonitrile less than about 10 ppm.

Nicotinamide Riboside Triacetate Chloride included a particle size determined by sieve analysis: Greater than 850 μm—0.07%, between about 850-425 μm—13.92%, between about 425-250 μm—47.78%, between about 250-180 μm—33.86%, between about 180-150 μm—0.29%, between about 150-125 μm—2.05%, between about 125-75 μm—1.16%, between about 75 - 0 μm—0.55%. Bulk density measured using USP method 786, Stage 6 Harmonization, Official Aug. 1, 2015.

### Example 3: NRTA-Cl Crystallization, Self Seeded Preparation of Nicotinamide Riboside Triacetate Chloride Crystals

Crude Nicotinamide Riboside Triacetate Chloride product (˜78 g) produced in a manner similar to Example 1 was added to a 1 L jacketed reactor set to about 20° C. To the crude product, about 83 g water and about 42 g ethanol was added. The resultant mixture was stirred and heated to about 30° C. to facilitate dissolution. Once in solution, about 907 g of additional ethanol was slowly metered into the reactor at about 10 mL/min. The reactor was then cooled to about −10° C. and held overnight or between about 8 and about 12 hours. In this method, crystal formation was first observed at about −7° C. About 61 g dried material (˜78% mass recovery, ˜85% yield adjusted for dry content & starting material purity) of a white, crystalline powder was obtained.

Crystalline Nicotinamide Riboside Triacetate Chloride included a purity determined by HPLC: ˜99.3%. Nicotinamide Riboside Triacetate Chloride included BRL<0.17% Nicotinamide.

Nicotinamide Riboside Triacetate Chloride included residual solvents determined by GC-MS: ˜3726.08 ppm Ethanol, Non-Detect Acetonitrile.

Nicotinamide Riboside Triacetate Chloride included a particle size determined by sieve analysis: Greater than 850 μm—0.04%, between about 850-425 μm—5.53%, between about 425-250 μm—39.32%, between about 250-180 μm—26.51%, between about 180-150 μm—9.43%, between about 150-125 μm—9.59%, between about 125-75 μm—7.47%, between about 75-0 μm—1.61%.

### Example 4: NRTA-Cl Crystallization, Seeded Preparation of Nicotinamide Riboside Triacetate Chloride Crystals

Crude Nicotinamide Riboside Triacetate Chloride product (˜577 g) produced in a manner similar to Example 1 was added to a 5 L jacketed reactor set to about 20° C. To the crude product, about 373 g water and about 191 g ethanol was added. The resultant mixture was stirred and heated to about 30° C. to facilitate dissolution. Once in solution, about 925 g of additional ethanol was slowly metered into the reactor at about 30 mL/min. The reactor was then cooled to about 10° C. Once at about 10° C., about 6 g of Nicotinamide Riboside Triacetate Chloride seed crystals produced in a manner similar to Example 3 was added into the reactor and allowed to isotherm for about 1 hour. Crystal formation was immediately observed following seed addition. Following an about 1-hour isotherm, an additional about 2,229 g of ethanol was slowly metered into the reactor at about 15 mL/min. At the onset of the second addition of ethanol, the reactor was first cooled to about 0° C. over about 200 minutes, then cooled to about −5° C. over about 50 minutes, and finally cooled to about −10° C. over about 20 minutes. The reactor was then cooled to about −10° C. and held overnight or between about 8 and about 12 hours. Excluding seed material, about 469 g dried material (˜81% mass recovery, ˜83% yield adjusted for starting material dry content & purity) of a white, crystalline powder was obtained.

Crystalline Nicotinamide Riboside Triacetate Chloride included a purity determined by HPLC: ˜100.7%. Nicotinamide Riboside Triacetate Chloride included ˜0.513% of Nicotinamide.

Nicotinamide Riboside Triacetate Chloride included residual solvents determined by GC -MS: ˜538.799 ppm Ethanol, Non-Detect Acetonitrile.

Nicotinamide Riboside Triacetate Chloride included a particle size distribution determined by sieve analysis: Greater than 850 μm—2.52%, between about 850-425 μm—44.76%, between about 425-250 μm—16.86%, between about 250-180 μm—11.16%, between about 180-150 μm—6.92%, between about 150-125 μm—8.14%, between about 125-75 μm—7.76%, between about 75-0 μm—1.69%.

### Example 5: NRTA-Cl Crystallization, Seeded Preparation of Nicotinamide Riboside Triacetate Chloride Crystals

Crude Nicotinamide Riboside Triacetate Chloride product (˜121 g) produced in a manner similar to Example 1 was added to a 1 L jacketed reactor set to about 20° C. To the crude product, about 72 g water and about 48 g ethanol was added. The resultant mixture was stirred and heated to about 30° C. to facilitate dissolution. Once in solution, about 168 g of additional ethanol was slowly metered into the reactor at about 10 mL/min. The reactor was then cooled to about 12.5° C. Once at about 12.5° C., about 1.2 g of Nicotinamide Riboside Triacetate Chloride seed crystals produced in a manner similar to Example 3 was added into the reactor and allowed to isotherm for about 1 hour. Crystal formation was immediately observed following seed addition. Following an about 1-hour isotherm, an additional about 431 g of ethanol was slowly metered into the reactor at about 5 mL/min. At the onset of the second addition of ethanol, the reactor was first cooled to about 0° C. over about 250 minutes, then cooled to about −5° C. over about 50 minutes, and finally cooled to about −10° C. over about 20 minutes. The reactor was then cooled to about −10° C. and held overnight or between about 8 and about 12 hours. Excluding seed material, about 104.5 g dried material (˜86% mass recovery, ˜89% yield adjusted for starting material dry content & purity) of a white, crystalline powder was obtained.

Crystalline Nicotinamide Riboside Triacetate Chloride included a purity determined by HPLC: ˜99.8%. Nicotinamide Riboside Triacetate Chloride included ˜0.348% of Nicotinamide.

Nicotinamide Riboside Triacetate Chloride included residual solvents determined by GC-MS: −294.615 ppm Ethanol, Non-Detect Acetonitrile.

Nicotinamide Riboside Triacetate Chloride included a particle size determined by sieve analysis: Greater than 850 μm—0.91%, between about 850-425 μm—21.57%, between about 425-250 μm—22.95%, between about 250-180 μm—21.57%, between about 180-150 μm—9.46%, between about 150-125 μm—10.27%, between about 125-75 μm—10.20%, between about 75 -0 μm—2.86%.

### Example 6: NRTA-Cl Crystallization, Seeded Preparation of Nicotinaide Riboside Triacetate Chloridie Crystals

Crude Nicotinamide Riboside Triacetate Chloride product (˜121 g) produced in a manner similar to Example 1 was added to a 1 L jacketed reactor set to about 20° C. To the crude product, about 72 g water and about 48 g ethanol was added. The resultant mixture was stirred and heated to about 30° C. to facilitate dissolution. Once in solution, about 168 g of additional ethanol was slowly metered into the reactor at about 10 mL/min. The reactor was then cooled to about 15° C. Once at about 15° C., about 1.2 g of Nicotinamide Riboside Triacetate Chloride seed crystals produced in a manner similar to Example 3 was added into the reactor and allowed to isotherm for about 1 hour. Crystal formation was immediately observed following seed addition. Following a 1-hour isotherm, an additional about 431 g of ethanol was slowly metered into the reactor at about 5 mL/min. At the onset of the second addition of ethanol, the reactor was first cooled to about 0° C. over about 300 minutes, then cooled to about −5° C. over about 50 minutes, and finally cooled to about −10° C. over about 20 minutes. The reactor was then cooled to about −10° C. and held overnight or between about 8 and about 12 hours. Excluding seed material, about 103.9 g dried material (˜86% mass recovery, ˜88% yield adjusted for starting material dry content & purity) of a white, crystalline powder was obtained.

Crystalline Nicotinamide Riboside Triacetate Chloride included a purity determined by HPLC: ˜99.2%. Nicotinamide Riboside Triacetate Chloride included ˜0.351% of Nicotinamide.

Nicotinamide Riboside Triacetate Chloride included residual solvents determined by GC-MS: −339.802 ppm Ethanol, Non-Detect Acetonitrile.

Nicotinamide Riboside Triacetate Chloride included a particle size determined by sieve analysis: Greater than 850 μm—1.14%, between about 850-425 μm—36.68%, between about 425-250 μm—18.08%, between about 250-180 μm—16.77%, between about 180-150 μm—9.86%, between about 150-125 μm—10.95%, between about 125-75 μm—5.77%, between about 75-0 μm—0.17%.

The use of the terms “a,” “an,” “the,” and similar referents in the context of describing the presently claimed invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments the values may range in value either above or below the stated value in a range of approximately ±5%; in other embodiments the values may range in value either above or below the stated value in a range of approximately ±2%; in other embodiments the values may range in value either above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof and, accordingly, reference should be made to the appended claims, rather than to the foregoing specification, as indicating the scope of the invention.

We claim:

1. A substantially crystalline compound having formula (VII), or a salt, or a solvate thereof: 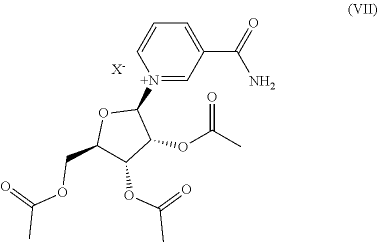
wherein X⁻ is a counterion;
the compound having a chemical purity of greater than about 90% (w/w) and containing less than about 5000 ppm ethanol.

2. The compound of claim 1 which is substantially a beta anomer form.

3. The compound of claim 1, wherein X⁻ is selected from the group consisting of fluoride, chloride, bromide, iodide, formate, acetate, propionate, butyrate, glutamate, aspartate, ascorbate, benzoate, carbonate, citrate, carbamate, gluconate, lactate, nitrate, phosphate, diphosphate, sulfate, succinate, sulfonate, trifluoromethanesulfonate, trichloromethanesulfonate, tribromomethanesulfonate, trichloroacetate, tribromoacetate, trifluoroacetate, malate, hydrogen malate, tartrate, hydrogen tartrate, glycolate, glucuronate, maleate, fumarate, pyruvate, anthranilate, 4-hydroxybenzoate, phenylacetate, mandelate, pamoate, methanesulfonate, ethanesulfonate, benzenesulfonate, panthothenate, 2-hydroxyethanesulfonate, p-toluenesulfonate, sulfanilate, cyclohexylaminosulfonate, stearate, palmitate, myristate, laurate, caprate, caprylate, caproate, oleate, linoleate, alginate, beta-hydroxybutyrate, salicylate, galactarate, and galacturonate.

4. The compound of claim 2, wherein X⁻ is chloride, having Form I.

5. The compound of claim 4, containing less than about 1000 ppm ethanol.

6. A nutritional supplement comprising any one of claims 1 to 5, including an excipient or a carrier.

7. A pharmaceutical composition comprising any one of claims 1 to 5, and a pharmaceutically acceptable carrier.

8. A method of making a substantially crystalline compound Nicotinamide Riboside Triacetate, or a salt, or a solvate thereof as in claim 1, comprising the steps of:
(a) adding a mass of Crude Nicotinamide Riboside Triacetate to a volume of a first solvent to form a reaction mixture;
(b) heating the reaction mixture to a temperature of about 20° C. to about 60° C.;
(c) cooling the reaction mixture;
(d) adding a second solvent; and
(e) isolating the substantially crystalline compound Nicotinamide Riboside Triacetate, or a salt, or a solvate thereof as a crystalline powder.

9. The method of claim 8, further comprising:
(b1) adding a third solvent immediately after step (b).

10. The method of claim 8, further comprising:
(c1) seeding the reaction mixture with crystalline compound Nicotinamide Riboside Triacetate, or a salt, or a solvate thereof after step (c).

11. The method of claim 10, wherein the crystalline compound Nicotinamide Riboside Triacetate is a chloride salt having Form I.

12. The method of claim 8, wherein the nicotinamide riboside solvate includes a solvent selected from the group consisting of water, acetic acid, acetone, acetonitrile, 1-butanol, 2-butanol, t-butyl alcohol, cyclohexane, 1,2-dichloroethane, diethylene glycol, diethyl ether, diglyme (diethylene glycol dimethyl ether), 1,2-dimethoxyethane, N,N-dimethylformamide, dimethylsulfoxide, 1,4-dioxane, ethanol, ethyl acetate, ethylene glycol, methanol (“MeOH”), methyl t-butyl ether, N-methyl-2-pyrrolidinone, 1-propanol, 2-propanol, pyridine, and tetrahydrofuran.

13. The method of claim 8, wherein the first solvent is selected from water, ethanol, or a mixture thereof.

14. The method of claim 8, wherein the second solvent is selected from one or more solvents selected from the group consisting of methyl t-butyl ether, acetone, methanol, ethanol, acetonitrile and water.

15. The method of claim 11, wherein the crystalline Nicotinamide Riboside Triacetate chloride Form I has a chemical purity of at least 99% at determined by HPLC.

16. A method for treating a condition that would benefit from increased intracellular NAD+ levels selected from conditions for treating and/or preventing symptoms, diseases, disorders, or conditions associated with, or having etiologies involving, vitamin B3 deficiency, indigestion, fatigue, canker sores, vomiting, poor circulation, burning in the mouth, swollen red tongue, depression, pellagra, Cockayne Syndrome, Neill-Dingwall Syndrome and progeria, in a subject mammal, comprising orally delivering to the mammal in need of such treatment an effective amount of a compound having formula (VII) according to claim 1.

17. The method of claim 16, wherein the compound having formula (VII) according to claim 1 is crystalline Nicotinamide Riboside Triacetate chloride having Form I.

18. A method of preparing an aqueous solution of crystalline Nicotinamide Riboside Triacetate chloride having Form I comprising providing a crystalline Nicotinamide Riboside Triacetate chloride compound having Form I, and contacting the compound with water.
